(19) United States
(12) Patent Application Publication
(10) Pub. No.: US 2026/0306273 A1
(43) Pub. Date: Oct. 8, 2026

(54) CONSTRAINED LINER FOR A PROSTHETIC JOINT

(71) Applicant: ENCORE MEDICAL, LP (D/B/A/ DJO SURGICAL), Austin, TX (US)

(72) Inventors: Adam David Shallenberg, Bentonville, AR (US); Alexander Horia Artaki, Austin, TX (US)

(21) Appl. No.: 19/636,816

(22) Filed: Apr. 1, 2026

Related U.S. Application Data
(60) Provisional application No. 63/782,428, filed on Apr. 2, 2025.

(51) Int. Cl. A61F 2/46; A61F 2/30; A61F 2/32; A61F 2/34

(52) U.S. Cl. CPC A61F 2/4609; A61F 2/30721; A61F 2/34; A61F 2002/30495; A61F 2002/3241

(57) ABSTRACT

An improved liner for a prosthetic joint is provided. The liner may include expansion slots at various intervals to allow the liner to expand to receive a prosthetic head. A channel extends around the circumference of the liner and is configured to receive a locking ring. When the locking ring is inserted into the channel, the liner is prevented from expanding, thereby securing the prosthetic head in the liner. The channel is advantageously configured to significantly reduce the difficulty of inserting the locking ring into the channel and removing the locking ring from the channel.

### CROSS REFERENCE TO RELATED APPLICATIONS

This application claims priority to and the benefit of U.S. Provisional Patent Application No. 63/782,428, filed Apr. 2, 2025, which is hereby incorporated by reference in its entirety.

### FIELD

This disclosure relates to a hip replacement implant and, more specifically, a constrained liner for an acetabular cup designed to receive a femoral stem implant therein.

### BACKGROUND

The human body contains many movable or articulating joints that allow at least two bone segments to move relative to each other when a force is applied. These joints also help maintain proper bone alignment, ensuring normal and efficient joint function. However, over time, various joints may become injured or experience wear and tear, affecting their ability to function properly. As a result, the bones may shift out of alignment or fail to maintain their natural positioning. Additionally, soft tissues play a crucial role in stabilizing the joints. Over time, these tissues can also become damaged or injured, impairing their ability to hold the joint in place. When this happens, the joint is more likely to become dislocated, and repeated dislocations can cause further damage to the joint and reduce the soft tissue's ability to stabilize it. In such cases, joint reconstruction is typically necessary.

Different types of prosthetic joints exist. These prosthetic joints may be implanted into a patient as an artificial replacement for the patient's damaged existing joint. As an example, a hip prosthesis (artificial hip joint) may include an acetabular component, a stem and head, and a liner. The acetabular component replaces the hip's natural socket, known as the acetabulum. The stem may be inserted into the femur. The head may be the ball-shaped part that replaces the natural ball of the femur. The head may fit into the acetabular component. The liner may sit inside the acetabular component and function as a bearing surface for the femoral head.

### SUMMARY

In one aspect, a prosthetic joint assembly is provided. The prosthetic joint assembly may include a prosthetic head, a liner configured to receive the prosthetic head, and an acetabular cup configured to receive the liner. The liner may include a channel disposed around a circumference thereof, the channel being configured to receive a locking ring therein. An upper portion of the liner has two or more segments configured to facilitate expansion of the upper portion of the liner so that the liner may receive the prosthetic head therein. The locking ring has an attachment portion having a substantially circular cross-section that is configured to be received within a jaw extending from an upper portion of the liner. The jaw may define an opening therein configured to receive the attachment portion of the locking ring, where the opening has a substantially circular cross-section and is a portion of the channel. The liner and the locking ring may have a pre-assembled position where the attachment portion of the locking ring is secured within the channel. In the pre-assembled position, the attachment portion of the locking ring is secured within the opening of the jaw of the liner, but the locking ring is not entirely secure within the channel. When the liner and the locking ring are in the pre-assembled position, the prosthetic head may be inserted into the liner. The locking ring may be configured to be secured within the channel when the prosthetic head is placed within the liner to prevent the prosthetic head from being displaced from the liner. The locking ring may be configured to prevent circumferential expansion of the liner when the locking ring is secured within the channel. In some aspects, the liner may be pre-assembled with the acetabular cup.

In some aspects, the locking ring, except for the attachment portion, has a substantially non-circular cross-section. In some aspects, the locking ring, when secured within the channel, extends upward from the channel to a top rim of the liner. In some aspects, the channel, except for the opening, has a substantially non-circular cross-section, and the opening defined in the jaw is configured to receive a portion of the locking ring having a substantially circular cross-section. In some aspects, at least a portion of the liner has an extended face. The extended face may be provided at an angle of about 10 degrees.

In another aspect, a liner for a prosthetic joint assembly is provided. The liner includes a channel for receiving a locking ring for securing a prosthetic head within the liner, where the channel has a first circular portion and a second non-circular portion. The first circular portion is configured to receive a reciprocal circular portion of the locking ring for providing the liner and the locking ring in a pre-assembled state. An upper portion of the liner may have two or more segments configured to facilitate expansion of the upper portion of the liner so that the liner may receive the prosthetic head therein. When the liner and the locking ring are in the pre-assembled configuration, the prosthetic head may be inserted into the liner. The locking ring may be configured to be secured within the channel when the prosthetic head is placed within the liner to prevent the prosthetic head from being displaced from the liner. The locking ring is configured to be secured within the channel when the prosthetic head is placed within the liner to prevent the prosthetic head from being displaced from the liner. When the liner and the locking ring are in the pre-assembled position, the prosthetic head may be inserted into the liner. The locking ring may be configured to be secured within the channel when the prosthetic head is placed within the liner to prevent the prosthetic head from being displaced from the liner. The locking ring may be configured to prevent circumferential expansion of the liner when the locking ring is secured within the channel.

In yet another aspect, a method for assembling a prosthetic joint is provided. The method includes the steps of providing an acetabular cup, inserting a liner into the acetabular cup, where the liner is configured to be secured within the acetabular cup using a plurality of protrusions configured to be received within reciprocal openings within the acetabular cup, providing a locking ring configured to be received within a channel disposed around the circumference of the liner, securing an attachment portion of the locking ring within an opening of the channel, such that the liner and the locking ring are provided in a pre-assembled position, inserting a prosthetic head into the liner, and securing the locking ring within the channel to retain the prosthetic head within the liner.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. In the drawings, the left-most digit(s) of a reference numeral may identify the drawing in which the reference numeral first appears. The use of the same reference numerals indicates similar, but not necessarily the same or identical components. However, different reference numerals may be used to identify similar components as well. Various embodiments may utilize elements or components other than those illustrated in the drawings, and some elements and/or components may not be present in various embodiments. The use of singular terminology to describe a component or element may depend on the context, encompass a plural number of such components or elements and vice versa.

FIG. 1 is an exploded view of an acetabular cup and liner assembly with a locking ring, according to one or more embodiments of the present disclosure.

FIG. 2A is a perspective view of an acetabular cup and liner assembly with a locking ring in a pre-assembled position, according to one or more embodiments of the present disclosure.

FIG. 2B is a cross-sectional view of the acetabular cup and liner assembly and locking ring in the pre-assembled position of FIG. 2A, according to one or more embodiments of the present disclosure.

FIG. 2C is a perspective view of the acetabular cup and liner assembly and locking ring in the pre-assembled position of FIG. 2A, with an inserted femoral head, according to one or more embodiments of the present disclosure.

FIG. 3A is a perspective view of an acetabular cup and liner assembly with a locking ring and inserted femoral head in an assembled position, according to one or more embodiments of the present disclosure.

FIG. 3B is a cross-sectional view of the acetabular cup and liner assembly with the locking ring and inserted femoral head in the assembled position of FIG. 3B, according to one or more embodiments of the present disclosure.

FIG. 4A is an exploded view of an acetabular cup and face change liner assembly with a locking ring and inserted femoral head in an assembled position, according to one or more embodiments of the present disclosure.

FIG. 4B is a front view of the acetabular cup and face change liner assembly with the locking ring and inserted femoral head in the assembled position of FIG. 4A, according to one or more embodiments of the present disclosure.

FIG. 4C is a cross-sectional view of the acetabular cup and face change liner assembly with the locking ring and inserted femoral head in the assembled position of FIG. 4A, according to one or more embodiments of the present disclosure.

FIG. 5 is an exploded view of an acetabular cup and liner assembly with a locking ring, according to one or more embodiments of the present disclosure.

FIG. 6 is a front view of an acetabular cup and liner assembly with a locking ring and inserted femoral head in a pre-assembled position, according to one or more embodiments of the present disclosure.

FIG. 7 is a front view of an acetabular cup and liner assembly with a locking ring and inserted femoral head in an assembled position, according to one or more embodiments of the present disclosure.

FIG. 8 is a graph comparing the force required to remove a femoral head from locking ring and liner assemblies, according to one or more embodiments of the present disclosure.

### DETAILED DESCRIPTION

This disclosure relates to, among other things, an improved liner (such as a constrained liner, for example) for a prosthetic joint. As one example, the prosthetic joint may be a hip prosthesis, however, other types of prosthetic joints may also be applicable. The improved liner includes a channel configured to receive a locking ring. When fully inserted into the channel, the locking ring prevents the liner from expanding, thereby preventing a prosthetic head inserted into the liner (which is provided in an acetabular cup of the prosthesis) from dislodging from the liner after the implant is fully assembled within the patient. This ensures that the prosthetic remains intact and operational for the patient after the surgical procedure.

While the locking ring provides an effective mechanism for securing the prosthetic head within the liner, there may exist some circumstances under which it is desired for the prosthetic head to be removed from the liner. For example, it may be desired to remove the prosthetic head (or disassemble any other portion of the prosthetic or all of the prosthesis) for maintenance, insert a new prosthesis into the patient, or perform any other tasks. Given the possibility that it may be desired to remove the prosthetic head from the liner, the channel on the liner is specifically configured to significantly reduce the difficulty of the processes of inserting and removing the locking ring into and out of the channel.

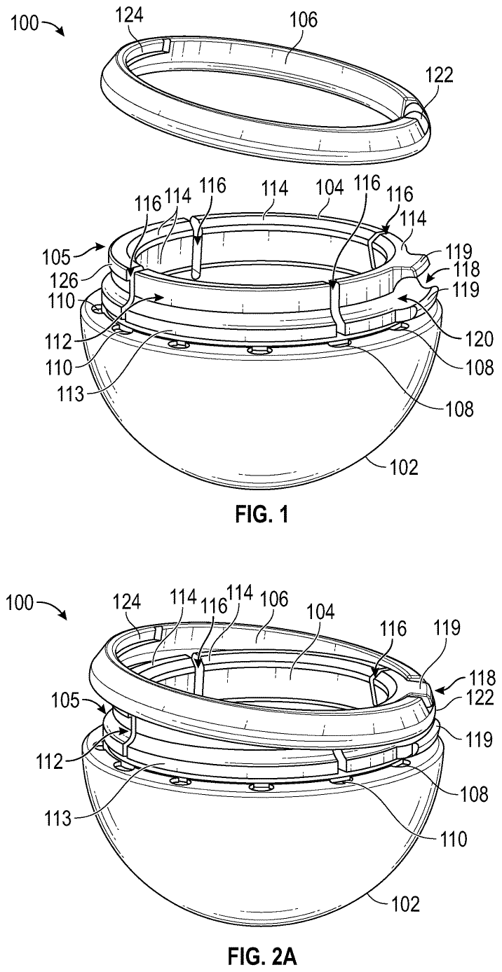

Referring now to the drawings, FIGS. 1-3B depict a subset of components included in a prosthetic hip joint assembly 100, as described in greater detail below. As shown in FIG. 1, the prosthetic hip joint assembly 100 may include an acetabular cup 102 (also generally referred to as a “shell” or the like), a liner 104, and a locking ring 106. When assembled, the acetabular cup 102, the liner 104, and the locking ring 106 may serve as the “socket” of the prosthetic joint configured to receive a prosthetic head (not shown) that serves as the “ball” in the prosthetic joint.

In embodiments, the acetabular cup 102 may be fixed to a boney structure at the joint of the patient, or secured in any other manner within the patient's body. The acetabular cup 102 includes an internal cavity configured to receive the liner 104 therein. In some embodiments, the acetabular cup 102 includes a plurality of openings 108 around the perimeter thereof configured to receive reciprocal protrusions 110 extending from the liner 104 around the circumference thereof. The engagement between the openings 108 of the acetabular cup 102 and protrusions 110 of the liner 104 may further secure the liner 104 within the acetabular cup 102.

According to preferred embodiments, the liner 104 also includes a channel 112 that is configured to receive the locking ring 106 therein. As used herein, a “channel” is a depression within the liner 104 that is capable of receiving the locking ring 106 therein. The channel 112 may be bounded on one or more sides by protrusions that define the depression within the liner 104 (i.e., the channel 112 may be bounded on the bottom side, the top side, or both sides to define the depression within the liner 104), and may be continuous or discontinuous around the circumference of the liner 104 (i.e., the depression forming the channel 112 may extend around the entire circumference of the liner 104 or may have breaks or interruptions therein such that the depression is not formed around the entire circumference of the liner 104). In some embodiments, the locking ring 106 is continuous, and extends around the entire circumference of the liner 104 without any interruptions or breaks therein. In other embodiments, the locking ring 106 is not continuous and has one or more breaks or interruptions, or does not extend around the entire circumference of the liner 104. With the liner 104 being unable to expand, the prosthetic head is unable to dislodge from and remains secure within the liner 104.

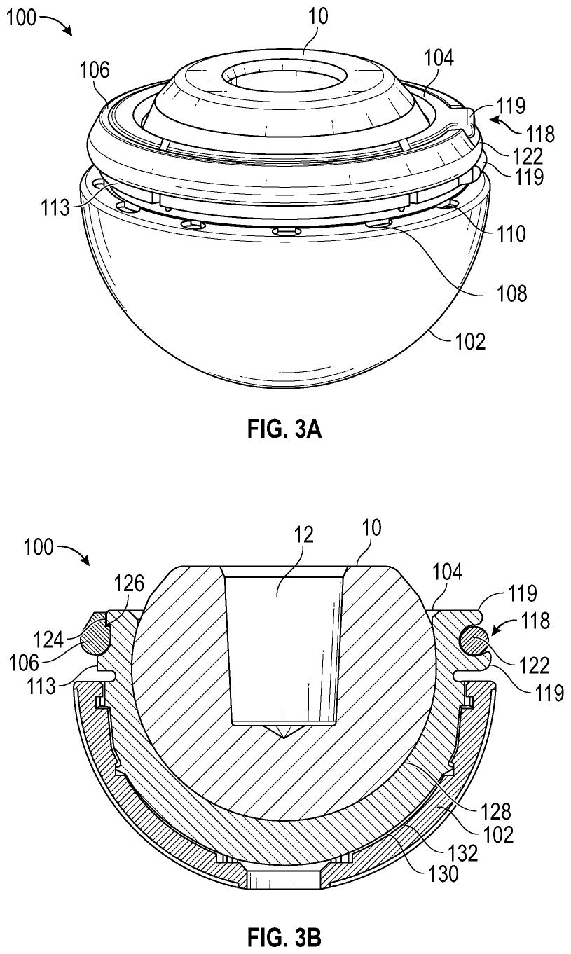

The locking ring 106 may be provided within the channel 112 around the circumference of the liner 104 and prevents the liner 104 from expanding. As shown in FIG. 1, the channel 112 may be disposed within an upper portion 105 of the liner 104 that protrudes from the cavity of the acetabular cup 102. In embodiments, the channel 112 is bounded by a lower lip 113 which may support the lower edge of the locking ring 106 so that it is properly positioned relative to the liner 104 and does not become displaced. According to preferred embodiments, the channel 112 is only bounded by the lower lip 113, with the opposing end of the channel being unbounded, such that the locking ring 106 covers the entire distance between the lower lip 113 and the top of the liner 104, as described in greater detail with respect to FIGS. 3A-3B. However, in some embodiments, such as those shown with respect to FIGS. 5-7B, the channel 112 may also be bounded by an upper lip (not shown) or other feature designed to retain the locking ring 106 within the channel 112.

In embodiments, the upper portion 105 of the liner 104 is castellated, having a plurality of segments 114 separated by notches 116 which allow the upper portion 105 of the liner 104 to compress under inwardly applied force by the locking ring 106 (and, conversely, expand when the locking ring 106 is not present or in the unlocked position). In some embodiments, the notches 116 are spaced evenly around the circumference of the upper portion 105 of the liner 104, defining uniformly sized segments 114 therebetween. In other embodiments, the notches 116 are not evenly spaced and the segments 114 are not uniformly sized.

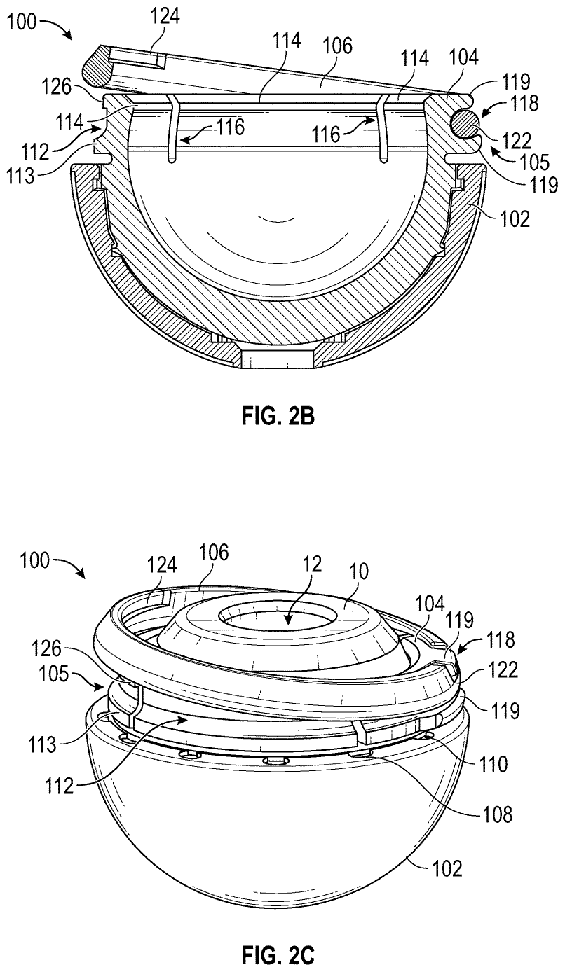

The upper portion 105 of the liner 104 may also include a jaw 118 configured to receive the locking ring 106 therein, so that the locking ring 106 may be in a pre-assembled position, as shown in FIGS. 2A-2B. In embodiments, the jaw 118 includes a pair of arms 119 defining an opening 120 therebetween, where the opening 120 is the portion of the channel 112 that passes between the arms 119 of the jaw 118. In some preferred embodiments, the opening 120 has a substantially circular cross section, where the remainder of the channel 112 has a non-circular cross-section. In these embodiments, the ring 106, as shown for example in FIG. 1, has an attachment portion 122 having a circular cross section that is configured to be received within the opening 120 of the jaw 118. In some embodiments, the remainder of the ring 106 has a non-circular cross section. In other embodiments, the remainder of the ring 106 also has a circular cross section.

In embodiments, the jaw 118 secures the locking ring 106 relative to the liner 104 so that the locking ring 106 can be “pre-assembled” with the liner 104 before the joint assembly 100 (or a portion thereof) is placed within a patient. In this pre-assembled position, as shown in FIGS. 2A-2C, the locking ring 106 is able to pivot relative to the liner 104. Once the prosthetic head is in place within the liner 104, the locking ring 106 can be manipulated and pivoted down towards the channel 112 of the liner 104 until it is locked into place. While the opening 120 is described herein as being circular, or substantially circular, it would be understood that that opening 120 may have any size or shape so long as the attachment portion 122 of the locking ring 106 is capable of being received and secured therein.

The ring 106 may also include a recess 124 on an interior surface thereof, the recess 124 being configured to engage with a reciprocal protrusion 126 extending from one of the segments 114 of the upper portion 105 of the liner 104. In some embodiments, the recess 124 is positioned on the ring 106 opposite from the attachment portion 122 thereof, with the reciprocal protrusion 126 being positioned opposite the jaw 118 on the upper portion 105 of the liner 104. However, it is not necessary that the recess 124 and the reciprocal protrusion 126 be opposite the attachment portion 122 and jaw 118, respectively. The reciprocal protrusion 126 may extend from any segment 114 of the upper portion 105 of the liner 104, except the segment 114 having the jaw 118, and the recess 124 is positioned at a corresponding position around the circumference of the ring 106.

When the ring is moved from the unlocked or preassembled position to the locked position, the downward force of the ring may force one or more segments of the upper portion of the liner inward so that the ring may fit within the channel. Once the ring is lowered, engagement between the recess and the reciprocal protrusion help to keep the ring in place relative to the liner. In preferred embodiments, all the segments except one are configured to be stationary or substantially stationary, with one segment that moves relative to the other segments. This allows for flexibility when inserting the head into the liner. But once the head is inserted into the liner, the liner should be compressed around the head with the help of the ring, which forces this segment in and holds it locked. In some other embodiments, all the segments may be able to move to achieve essentially the same purpose.

Referring now to FIGS. 2A-2C, the prosthetic hip joint assembly 100 may be provided in a pre-assembled position where the liner 104 is inserted into the acetabular cup 102, and the locking ring 106 is at least partially connected to the liner 104. For example, in preferred embodiments such as that shown in FIGS. 2A-2B, the attachment portion 122 of the locking ring 106 is held within the opening 120 of the jaw 118, so that the locking ring 106 is partially disposed within the channel 112 of the upper portion 105 of the liner 104. The jaw 118, specifically the arms 119 thereof, may maintain the locking ring 106 in this preassembled position, with a portion of the locking ring 106 being outside of the channel 112 and protruding above the liner 104. With the locking ring 106 only partially inserted into the channel 112, the liner 104 can still expand, via movement of one or more segments 114 of the upper portion 105 of the liner, to receive the prosthetic head. Once the prosthetic head is inserted into the liner 104, as shown in FIG. 2C, the locking ring 106 may be fully inserted into the channel 112 by providing a force on the portion of the locking ring 106 that is outside of the channel 112 to press the locking ring 106 fully into the channel 112. This results in a more seamless installation process for the prosthetic joint. The prosthetic joint assembly 100 may be provided with the locking ring 106 pre-attached to the liner 104 (i.e., the liner 104 and the locking ring 106 may be assembled during manufacturing), or the locking ring 106 may be attached to the liner 104 in the operating room prior to implanting the prosthetic joint in the patient. It is also possible to attach the locking ring 106 to the liner after the acetabular cup 102 and liner 104 have been implanted within the patient's joint.

FIGS. 3A-3B depict the prosthetic joint assembly 100 with the locking ring 106 in the assembled position, where the locking ring 106 is fully inserted into the channel 112. With the locking ring 106 inserted into the channel 112, the liner 104 is prevented from expanding, thereby securing the prosthetic head 10 in place with the liner 104 and the acetabular cup 102 to prevent the prosthetic head 10 from unintentionally dislodging from the liner 104 after the prosthetic is implanted in the patient. In addition to facilitating insertion of the prosthetic head 10, the segmented design of the upper portion 105 of the liner 104 may allow the surgeon, or other personnel, to more easily insert and remove the locking ring 106 to and from the channel 112. For example, to remove the locking ring 106, an operator may force at least one movable segment 114 of the liner 104 towards the center thereof, and then pull up on the locking ring 106 to displace it from the channel 112.

According to preferred embodiments, as shown in FIGS. 3A-3B, the locking ring 106 is sized and shaped so that, when the locking ring 106 is in the assembled or locked position, the locking ring 106 covers the entire top portion 105 of the liner 104 above the lip 113 bounding the channel 112. Because the locking ring 106, in preferred embodiments, has a non-circular cross-section except for the attachment portion 122 of the ring 106, the ring 106 may cover a larger portion of the liner 104 as compared to existing prosthetic joints, thereby improving the strength of the assembly 100 as shown and described herein. Without intending to be bound by a particular theory, it is believed that the increased surface contact between the ring 106 and the liner 104 provides better security for the prosthetic head 10, such that it is less likely that the prosthetic head 10 will become unintentionally dislodged from the liner 104.

In embodiments, as shown in FIG. 3B, the liner 104 can define an internal cavity or arcuate region 128. The internal arcuate region 128 can define a portion or a hemisphere and can also include the upper portion 105 of the liner 104 that extends above the hemisphere. The liner 104 can further define an exterior arcuate portion 130. The exterior arcuate portion 130 can include a portion that substantially engages or mates with an internal arcuate portion 132 of the acetabular cup 102 (for example, when the liner 104 is inserted into the acetabular cup 102 during assembly of the full prosthetic joint). Although the exterior portion 130 of the liner 104 does not necessarily need to contact the entire interior region 132 of the acetabular cup 102, the exterior portion 130 can be provided to contact at least a portion thereof to distribute a force.

In one or more embodiments, the prosthetic head 10 may serve as the “ball” in the prosthetic ball and socket mechanism formed by the prosthetic joint when assembled. For example, the prosthetic head 10 may be substantially spherical in shape (however, other shapes are also possible). The prosthetic head 10 can define a recess 12 for engaging or interconnecting with the stem (not shown). While the recess 12 is shown as being a particular shape and size within the prosthetic head 10, one of ordinary skill in the art would appreciate that the recess may be any other size and/or shape depending on the configuration of the stem received by the prosthetic head.

In one or more embodiments, the stem may be the portion of the prosthetic joint that is inserted into the femur (thigh bone) of the patient. For example, the stem can be made of metal (titanium or cobalt-chromium alloys, etc.), and its primary function is to anchor the prosthesis securely in the femur. One end of the stem is inserted into the femur and the other end of the stem is inserted into a cavity within the prosthetic head.

In one or more embodiments, the acetabular cup is placed in a prepared acetabulum of the patient. Extending from the exterior of the acetabular cup may be spikes or projections that may be used to embed the acetabular cup into the acetabulum. In addition, various holes may be provided to allow fasteners (screws, etc.) to be placed through the acetabular cup to be received in the acetabulum. The acetabular cup may also be secured within the acetabulum using any other suitable mechanism. Additionally, in some instances, the acetabular cup may be secured within the acetabulum using an adhesive or other compound.

The interior of the acetabular cup may include a cavity. The cavity, in one or more embodiments, may be substantially hemispherical (however, other shapes are also possible). The cavity of the acetabular cup may be sized and shaped to at least partially receive the prosthetic head. In some embodiments, the prosthetic joint may not necessarily require the acetabular cup and the liner may instead be directly attached to the acetabulum of the patient.

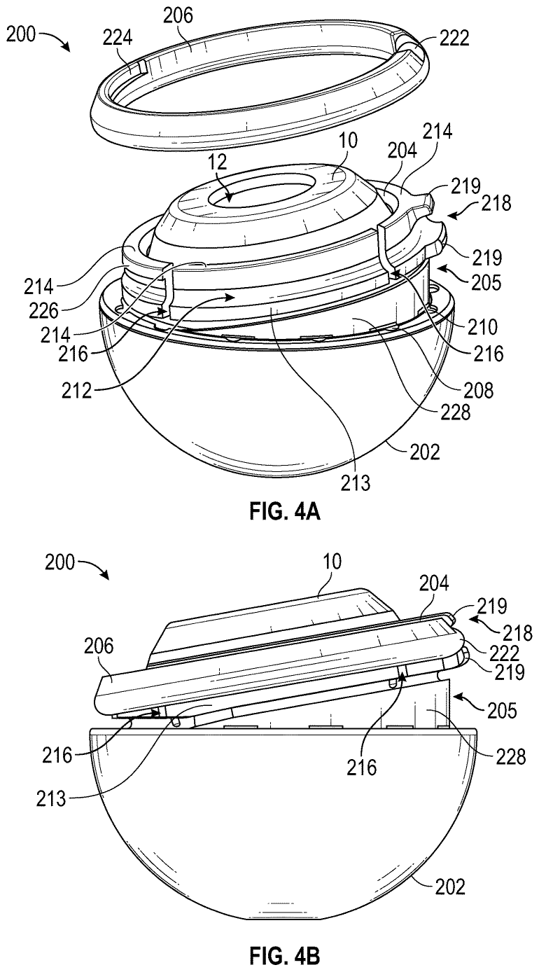

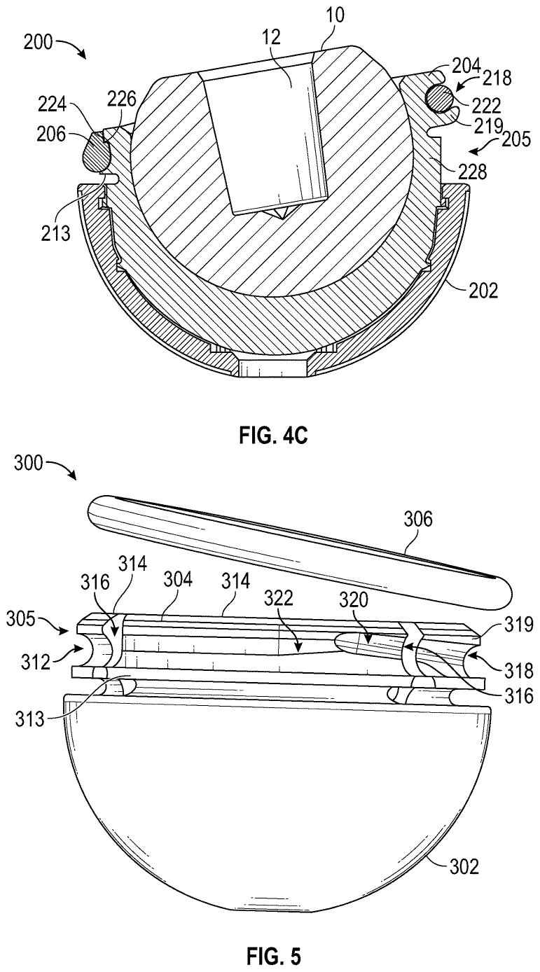

In embodiments, as shown in FIGS. 4A-4C, a face changer prosthetic joint assembly 200 may be provided to permit the surgeon to change the angle, orientation, or position of the prosthetic joint assembly 200 within the patient's joint socket. Like the assembly 100 of FIGS. 1A-3B, the joint assembly 200 includes an acetabular cup 202, a liner 204, and a locking ring 206. In some embodiments, the acetabular cup 202 includes a plurality of openings 208 around the perimeter thereof configured to receive reciprocal protrusions 210 extending from the liner 204 around the circumference thereof. The engagement between the openings 208 of the acetabular cup 202 and protrusions 210 of the liner 204 may further secure the liner 204 within the acetabular cup 202.

The liner 204 may also include a channel 212 configured to receive the locking ring 206 therein, which is disposed within an upper portion 205 of the liner 204 that protrudes from the cavity of the acetabular cup 202. The channel 212 may be bounded by a lower lip 213, which may abut and hold the ring in place so that it is properly positioned and does not slide about the liner. According to preferred embodiments, the channel 212 is only bounded by the lower lip 213, with the opposing end of the channel being unbounded, such that the locking ring 206 takes up the entire space of the upper portion of the liner between the lower lip 213 and the top of the liner 204.

The upper portion 205 of the liner 204 may also be castellated, having a plurality of segments 214 separated by notches 216 which allow the upper portion 205 of the liner 204 to compress under inwardly applied force by the locking ring 206 (and, conversely, expand when the locking ring 206 is not present or in the locked position). The notches 216 may be spaced evenly around the circumference of the upper portion 205 of the liner 204, defining uniformly sized segments 214 therebetween, or the notches 216 may not be evenly spaced and the segments 214 are not uniformly sized.

The upper portion 205 of the liner 204 may also include a jaw 218 configured to receive the locking ring 206 therein, so that the locking ring 206 may be in a pre-assembled position (not shown). In embodiments, the jaw 218 includes a pair of arms 219 defining an opening 220 therebetween, where the opening 220 is the portion of the channel 212 that passes between the arms 219 of the jaw 218. In some preferred embodiments, the opening 220 has a substantially circular cross section, where the remainder of the channel 212 has a non-circular cross-section. In these embodiments, the ring 206 has an attachment portion 222 having a circular cross section that is configured to be received within the opening 220 of the jaw 218. In some embodiments, the remainder of the ring 206 has a non-circular cross section. In other embodiments, the remainder of the ring 206 also has a circular cross section.

The ring 206 may also include a recess 224 on an interior surface thereof, the recess 224 being configured to engage with a reciprocal protrusion 226 extending from one of the segments 214 of the upper portion 205 of the liner 204. In some embodiments, the recess 224 is positioned on the ring 206 opposite from the attachment portion 222 thereof, with the reciprocal protrusion 226 being positioned opposite the jaw 218 on the upper portion 205 of the liner 204. However, it is not necessary that the recess 224 and the reciprocal protrusion 226 be opposite the attachment portion 222 and jaw 218, respectively. The reciprocal protrusion 226 may extend from any segment 214 of the upper portion 205 of the liner 204, except the segment 214 having the jaw 218, and the recess 224 is positioned at a corresponding position around the circumference of the ring 206.

In these embodiments, the upper portion 205 of the liner 204 may also have an extended face 228. The extended face 228 may help the surgeon to fine-tune the orientation of the prosthetic joint assembly 200, particularly when there is a need to correct or optimize the inclination and anteversion of the assembly 200 when anatomical deficiencies or other surgical constraints are present. The extended face 228 may therefore provide an angular offset between the liner 204 and the acetabular cup 202, i.e., the extended face 228 reorients the liner 204 relative to the cup 202. In this way, the bearing surface may sit at a more optimal angle while still maintaining the appropriate coverage of the acetabular cup 202 within the joint.

To create this offset, and the extended face, the lower lip 213 of the liner 204 may be angled relative to the acetabular cup 202. In some embodiments, the angle of the lower lip 213 relative to the acetabular cup is between about 5 degrees and about 20 degrees, preferably about 10 degrees. While the extended face 228 is shown in FIGS. 3A-3B as having a maximum distance from the acetabular cup 202 at the same position around the circumference of the liner as the jaw 218, it would be understood that the extended face 228 may be provided in any orientation relative to the features of the assembly 200.

Referring now to FIGS. 5-7, another embodiment of a prosthetic joint assembly is shown. Like the assembly of FIGS. 1A-3B, the prosthetic joint assembly 300 includes an acetabular cup 302, a liner 304, and a locking ring 306. The acetabular cup 302 may be fixed to a bony structure at the joint of the patient and may include an internal cavity configured to receive the liner 304. The liner 304 also includes an internal cavity and is configured to receive a prosthetic head therein and may temporarily expand to do so. In some embodiments, the acetabular cup 302 includes a plurality of openings 308 around the perimeter thereof configured to receive reciprocal protrusions 310 extending from the liner 304 around the circumference thereof. The engagement between the openings 308 of the acetabular cup 302 and protrusions 310 of the liner 304 may further secure the liner 304 within the acetabular cup 302.

As shown in FIG. 5, the liner 304 may have an upper portion 305 that protrudes beyond the acetabular cup 302 and includes the channel 312 that is configured to receive the locking ring 306. The upper portion 305 of the liner 304 may include one or more features that allow the liner 304 to expand and contract to allow the prosthetic head to be inserted into and removed from the liner 304. Specifically, the upper portion 305 of the liner 304 may include one or more segments 314, separated by expansion slots 316 provided at various intervals around the upper portion 305 of the liner 304. The expansion slots 316 are gaps in the material of the liner 304 that provide space for the material of the liner 304 to expand and contract during insertion or removal of the prosthetic head. However, when the locking ring 306 is fully inserted into the channel 312 and around the upper portion 305 of the liner 304, the locking ring 306 prevents the upper portion 305 of the liner 304 from expanding outward.

In embodiments, the liner 304 includes a channel 312 that only partially extends around the circumference of the upper portion 305 of the liner 304. In one or more embodiments (although not visible), the channel 312 may partially wrap around the upper portion 305 of the liner 304 from a first end 318 to a second end 320 thereof. However, in other embodiments, multiple separate channels may be provided around the upper portion 305 of the liner 304. That is, rather than including one continuous channel, a first channel may wrap around a first portion of the liner 304 and a separate second channel may wrap around a second portion of the liner 304 (or any other number of distinct channels may be provided around the liner 304).

Given that the channel 312 does not extend around the full circumference of the upper portion 305 of the liner 304, the upper portion 305 includes a solid region 322 between the first end 318 of the channel 312 and the second end 320 of the channel 318. The solid region 322 generally refers to a non-concave region of the exterior surface of the upper portion 305 (or less concave than the regions where the channel is located 312). When the locking ring (for example, locking ring 306 or any other locking ring) is fully inserted into the channel 312 in the post-assembly position, a portion of the locking ring 306 rests against the surface of the solid region 322 rather than resting within the channel 312. This provides a location where a surgeon (or other personnel) can more effectively grasp the locking ring 306 to remove the locking ring 306 from the liner 304 (for example, if it is desired to remove the prosthetic head from the liner 304 to perform maintenance, to re-arrange the components of the prosthetic, or for any other reason).

If the channel 312 were to extend around the full circumference of the upper portion 305 of the liner 304, then the entirety of the locking ring 306 would be located within the channel 312 when the locking ring 306 is fully inserted, which may increase the difficulty of removing the locking ring 306 from the channel 312. That is, less of the locking ring 306 can protrude from the liner 304 when held within the recess formed by the channel 312, leaving less material for the surgeon to grasp to pull the locking ring 306 out of the channel 312. Additionally, the surgeon may be unable to (or may have difficulty) reaching underneath the locking ring 306 to obtain sufficient leverage to pull the locking ring 306 out of the channel 312. With the channel 312 only partially extending around the liner 304 and part of the locking ring 306 resting on the surface of the liner 304, the surgeon can more easily grasp the bottom of that portion of the locking ring 306 to obtain sufficient leverage to pull the locking ring 306 out of the channel 312.

In addition to the channel 312 only partially extending around the upper part 305 of the liner 304, at least a portion of the channel 312 is also provided at an angle. In other words, one part of the channel 312 may be at a first height on the upper portion 305 of the liner 304, another part of the channel 312 may be at a second height on the upper portion 305 of the liner 304, and the first height and the second height may be different (for example, the first height may be greater than the second height).

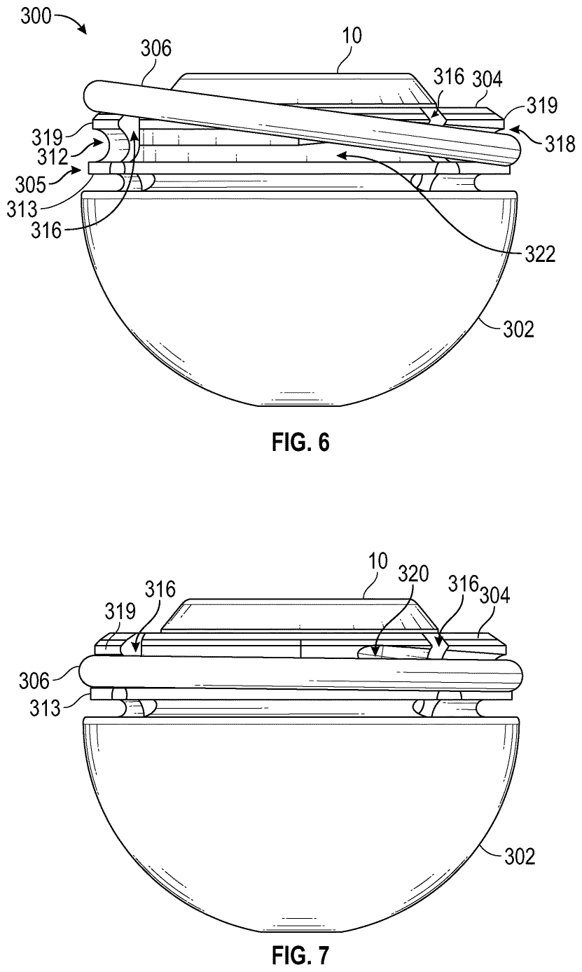

At least a portion of the channel 312 being provided at an angle further reduces the difficulty associated with the insertion and removal of the locking ring 306 to and from the channel and also allows for the locking ring 306 to rest in the pre-assembly position, as shown in FIG. 6, prior to being fully inserted into the channel 312. For example, a first portion of the locking ring 306 may be inserted on one side of the liner 304. When initially inserted, the angle of the channel 312 results in the locking ring being angled relative to the liner 304 such that a second portion of the locking ring protrudes above the liner 304 (this initial position of the locking ring is shown in FIG. 6). A force may then be provided to the second portion of the locking ring 306 to push the second portion of the locking ring 306 down into the channel 312 (at this point, the locking ring would be fully inserted into the channel 312 in the post-assembly position shown in FIG. 7). The channel 312 (or a portion of the channel 312) being provided at an angle also allows for the locking ring to rest in a pre-assembly position prior to being fully inserted into the channel (shown in FIG. 6). That is, the locking ring may be partially inserted into the channel 312 and the structure of the channel may support the locking ring in the pre-assembly position.

Additionally, in one or more embodiments, the diameter of the channel 312 may be non-uniform to further improve the locking ring 306 insertion and/or removal process. For example, the first end 318 of the channel 312 may have a smaller diameter than the second end 320 of the channel 312 (or the first end 318 of the channel 312 may have a smaller diameter than any other portion of the channel 312). The larger diameter at certain portions of the channel 312 provides additional space within the channel 312 for movement of the locking ring 306 (for example, vertical translation within the channel 312) when the locking ring 306 is fully inserted into the channel 312 or partially or fully removed from the channel 312.

Turning to FIG. 6, a side view of the acetabular cup 302, the liner 304, and the locking ring 306 are shown in a pre-assembly position. The pre-assembly position generally refers to the arrangement of the acetabular cup 302, the liner 304, and the locking ring 306 prior to the insertion of the prosthetic head into the liner 306. In the pre-assembly position, the liner 304 is inserted into the acetabular cup 302. The locking ring 306 may then be partially inserted into the channel 312 as shown in FIG. 6, for example. The angle of the channel 312 may also maintain the locking ring 306 in this pre-assembly position (with a portion of the locking ring 306 being outside of the channel 312 and protruding above the liner 304 as shown in FIG. 6). With the locking ring 306 only partially inserted into the channel 312, the liner 304 can still expand to receive the prosthetic head. Once the prosthetic head is inserted into the liner 304 (as shown in FIGS. 1A-1B), the locking ring 306 may easily be fully inserted into the channel 312 by providing a force on the portion of the locking ring 306 that is outside of the channel 312 (to press the locking ring 306 fully into the channel 312). This results in a more seamless installation process for the prosthetic joint. The locking ring 306 could be installed in-situ or could come out of the package in the pre-assembly position.

FIG. 7 depicts a side view of the acetabular cup 302 and liner 304 with the locking ring 306 in a post-assembly position. In the post-assembly position, the locking ring 306 is fully inserted into the channel 312. With the locking ring 306 fully inserted, the liner 304 is prevented from expanding, thereby securing the prosthetic head in place within the liner 304 and the acetabular cup 302 (to prevent the prosthetic head from unintentionally dislodging from the liner 304 after the prosthetic is implanted in the patient). In embodiments, the liner 304 is advantageously configured to allow for a surgeon (or other personnel) to more easily insert and remove the locking ring 306 into and from the channel 312. To remove the locking ring, an operator may “pull” a narrower flexible tab and then pull on the locking ring 306, for example.

In embodiments, when the locking ring 306 is in the pre-assembly position as shown in FIG. 6, the portion of the locking ring 306 that is inserted into the channel 312 is closer in proximity to the bottom 313 of the channel 312 than the top 319 of the channel 312, with a gap existing between the locking ring 306 and the top 319 of the channel 312. When the force is provided on the portion of the locking ring 306 that is outside of the channel 312 to fully insert the locking ring 306 into the channel 312, the locking ring 306 translates upwards within the channel 312 into the post-assembly position shown in FIG. 7. In the post-assembly position, the locking ring 306 is now closer in proximity to the top 319 of the channel 312 than the bottom 313 of the channel 312, with a gap existing between the locking ring 306 and the bottom 313 of the channel 312. The gaps may provide space for a first portion of the locking ring 306 that is initially inserted into the channel 312 during installation and removed last from the channel during removal to move within the channel 312. Accordingly, the first portion of the locking ring 306 can move within the channel 312 when the second portion of the locking ring 306 is pressed into the channel 312 during installation and when the second portion of the locking ring 306 is pulled out of the channel 312 during the removal process.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

### Examples

Four locking ring and liner assemblies were assessed for their capability in retaining a femoral head within a liner. The height of the ring and the interference between the ring and the liner were varied. The samples included two low wall samples, similar to existing locking rings that only partially cover the upper portion of the liner, one with no interference between the ring and the liner and one with interference between the ring and the liner, and two high wall samples, such as those described herein where the locking ring covers the entire upper portion of the liner, one with no interference between the ring and the liner and one with interference between the ring and the liner.

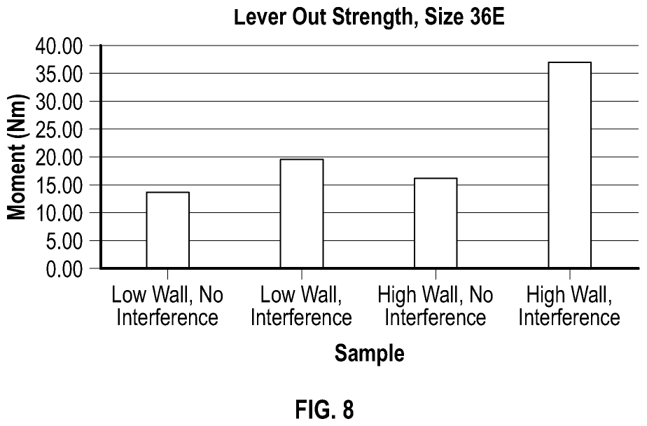

To test the capability of each locking ring and liner assembly, a lever-out test was performed where a levered force is applied to the femoral head to dislodge the head from the liner. The sample assembly with a high wall where there was interference between the ring and the liner performed the best, requiring nearly three times as much force to dislodge the femoral head from the liner as the low wall sample without interference between the ring and the liner, which was the worst performing sample. These results are shown in FIG. 8.

TABLE 1
Summary of force required to dislodge femoral head from
liner with each locking ring and liner assembly.

| Sample | Force (N) | Force (Nm) |
|---|---|---|
| Low Wall, No Interference | 180.4 | 13.53 |
| Low Wall, Interference | 258.8 | 19.41 |
| High Wall, No Interference | 214.4 | 16.08 |
| High Wall, Interference | 491.1 | 36.83 |

That which is claimed is:

1. A prosthetic joint assembly comprising:
a prosthetic head;
a liner configured to receive the prosthetic head; and
an acetabular cup configured to receive the liner;
wherein the liner comprises a channel disposed around a circumference thereof configured to receive a locking ring therein.

2. The prosthetic joint assembly of claim 1, wherein an upper portion of the liner comprises two or more segments configured to facilitate expansion of the upper portion of the liner so that the liner may receive the prosthetic head therein.

3. The prosthetic joint assembly of claim 1, wherein the locking ring comprises an attachment portion having a substantially circular cross-section that is configured to be received within a jaw extending from an upper portion of the liner.

4. The prosthetic joint assembly of claim 3, wherein the jaw defines an opening therein configured to receive the attachment portion of the locking ring, wherein the opening has a substantially circular cross-section, and wherein the opening is a portion of the channel.

5. The prosthetic joint assembly of claim 4, wherein the liner and the locking ring have a pre-assembled configuration where the attachment portion of the locking ring is secured within the opening of the jaw of the liner, but the locking ring is not entirely secure within the channel.

6. The prosthetic joint assembly of claim 5, wherein when the liner and the locking ring are in the pre-assembled configuration, the prosthetic head may be inserted into the liner.

7. The prosthetic joint assembly of claim 3, wherein the locking ring, except for the attachment portion, has a substantially non-circular cross-section.

8. The prosthetic joint assembly of claim 7, wherein the locking ring, when secured within the channel, extends upward from the channel to a top rim of the liner.

9. The prosthetic joint assembly of claim 4, wherein the channel, except for the opening, has a substantially non-circular cross-section, and wherein the opening defined in the jaw is configured to receive a portion of the locking ring having a substantially circular cross-section.

10. The prosthetic joint assembly of claim 1, wherein the locking ring is configured to be secured within the channel when the prosthetic head is placed within the liner to prevent the prosthetic head from being displaced from the liner.

11. The prosthetic joint assembly of claim 10, wherein the locking ring is configured to prevent circumferential expansion of the liner when the locking ring is secured within the channel.

12. The prosthetic joint assembly of claim 1, wherein the liner is configured to be pre-assembled with the acetabular cup.

13. The prosthetic joint assembly of claim 1, wherein at least a portion of the upper portion of the liner comprises an extended face.

14. The prosthetic joint assembly of claim 13, wherein the extended face is provided at an angle of about 10 degrees.

15. A liner for a prosthetic joint assembly, the liner comprising a channel disposed therein for receiving a locking ring for securing a prosthetic head within the liner, wherein the channel comprises a first circular portion and a second non-circular portion, wherein the first circular portion is configured to receive a reciprocal circular portion of the locking ring therein for providing the liner and the locking ring in a pre-assembled state.

16. The liner of claim 15, wherein an upper portion of the liner comprises two or more segments configured to facilitate expansion of the upper portion of the liner so that the liner may receive the prosthetic head therein.

17. The liner of claim 15, wherein when the liner and the locking ring are in the pre-assembled configuration, the prosthetic head may be inserted into the liner.

18. The liner of claim 15, wherein the locking ring is configured to be secured within the channel when the prosthetic head is placed within the liner to prevent the prosthetic head from being displaced from the liner.

19. The liner of claim 18, wherein the locking ring is configured to prevent circumferential expansion of the liner when the locking ring is secured within the channel.

20. A method for assembling a prosthetic joint comprising:
providing an acetabular cup;
inserting a liner into the acetabular cup, wherein the liner is configured to be secured within the acetabular cup using a plurality of protrusions configured to be received within reciprocal openings within the acetabular cup;
providing a locking ring configured to be received within a channel disposed around the circumference of the liner;
securing an attachment portion of the locking ring within an opening of the channel, such that the liner and the locking ring are provided in a pre-assembled position;
inserting a prosthetic head into the liner; and
securing the locking ring within the channel to retain the prosthetic head within the liner.
